(19) United States
(12) Patent Application Publication
(10) Pub. No.: US 2026/0177479 A1
(43) Pub. Date: Jun. 25, 2026

(54) IMPROVED NOISE CANCELING DETECTOR

(71) Applicant: Tunable AS, Oslo (NO)

(72) Inventors: Eivind Jülke Røer, Oslo (NO); Jon Olav GREPSTAD, Oslo (NO)

(73) Assignee: Tunable AS, Oslo (NO)

(21) Appl. No.: 19/128,324

(22) PCT Filed: Dec. 21, 2023
(86) PCT No.: PCT/NO2023/060131

§ 371 Date: May 8, 2025

(30) Foreign Application Priority Data
Dec. 22, 2022 (NO) 20221394

(51) Int. Cl. G01N 21/3504; G01N 21/17; G01N 21/39; G01N 27/407; G01N 33/00

(52) U.S. Cl. CPC G01N 21/3504; G01N 21/39; G01N 27/4071; G01N 33/0036; G01N 33/004; G01N 2021/1704; G01N 2021/399

(57) ABSTRACT

The present invention relates to a photoacoustic gas detector including a gas measuring volume and a gas reference volume where the volumes are separated by at least one flexible membrane being responsive to pressure differences between the volumes. The detector also comprises a light source emitting light in a predetermined wavelength range into the measuring volume, the wavelength range being chosen based on the absorption spectrum of a gas to be measured. The gas detector also includes a measuring unit for measuring membrane movements. The measuring and reference volumes both define known mass centers, the position of the mass centers being chosen so as to balance the pressure on the membrane when subject to a movement in at least one predetermined first direction.

The present invention relates to an optical gas detector for detecting a gas absorbing light at a known wavelength.

Absorption type gas sensors are well known where light is transmitted through a gas mixture towards a detector, where a certain gas absorbs light at certain characteristic wavelengths and if both the transmitted spectrum and the detected spectrum is known it is possible to see the concentration of the gas. Measuring the spectrum of the transmitted light is, however, a complicated process, and also difficult to incorporate in compact low-cost equipment such as devices for measuring alcohol content.

Some alternative solutions have been developed where the absorption of certain wavelengths in a gas may be utilized, e.g. where the absorption results in a rise of temperature in the gas, or in pressure fluctuations. The photoacoustic gas detectors invented by Brüel and Kjær (U.S. Pat. No. 4,818,882) have been demonstrated to detect very low levels of gas. The concept has been developed further, e.g. as described in WO2017/055219 describing a solution with symmetric membranes for reducing noise sensitivity, and also WO2017/089624. Breguet J et al.: “Photoacoustic detection of trace gases with an optical microphone” Sensors and Actuators A: Physical, Elsevier BV, NL, vol. 48, no. 1, 1 May 1995, describes the use of two membranes where one includes a fiber optic sensor. JP H01 277000 describes a solution with two symmetrically positioned microphones connected to closed containers. Other examples of photoacoustic sensors are discussed in EP1546684B1, EP3483589, U.S. Pat. Nos. 9,360,417 and 7,245,380, as well as in Kuusela et al; “Photoacoustic Gas Analysis Using Interferometric Cantilever Microphone”; Applied Science Spectroscopy Reviews, 42:5 443-474; 2007; DOI:10.1080/00102200701421755 and de Paula et al: “Optical microphone for photoacoustic spectroscopy”; Journal of applied physics 64, 3722/1988; DOI: 10.1063/1.341416.

Typically, the known solutions illuminate a volume of gas with pulsed electromagnetic radiation having a known wavelength corresponding to an absorption wavelength of the gas to be detected. The gas illumination is performed by aiming a light beam with a suitable wavelength and beam shape through a gas volume. If the gas is present each pulse heats the gas, generates a pressure wave applying a force on one or more membranes, beams, doors or similar which can be measured. The sensitivity will depend on the concentration of the gas as well as the geometry of the system coupling the illumination to the gas volume. It is an object of the present invention to provide a solution increasing the sensitivity of the measurements.

As discussed in WO2017/055219, two symmetric membranes side by side will reduce vibrational sensitivity for acceleration perpendicular to the membrane surface. However, when incorporated in closed volumes as proposed in WO2017/089624, vibrational sensitivity will be strongly dependent on the size and shape these volumes (e.g., the reference and measurement volume). It is therefore an object of the present invention to provide a robust photoacoustic instrument reducing the influence of vibrations and noise from the environment.

Thus, it is an object of the present invention to provide a compact and inexpensive photo acoustic gas sensor having low sensitivity to external vibrations while having high sensitivity gas detection. This is obtained with an optical gas detector characterized as stated in the accompanying claims.

The present invention is thus based on two principles for reducing the effect of vibrations and movement for an acousto-optical gas detector. The reference and gas volumes are balanced so that the membranes experience the same pressure on both sides of the membranes even though a movement will result in pressure variations in the gas volumes. In addition, the principle discussed in WO2017/055219 and WO2017/089624 may be used relati [symbol] case using two identical membranes.

This is performed by balancing the mass centers of the gas volumes. The definition of the mass center R(\vec{r}) for a volume V with a continuously distributed mass ρ(\vec{r}) and a total mass M is

$$R ( \vec{r} ) = \frac{1}{M} \int ρ ( \vec{r} ) \vec{r} dV .$$

If the mass density is uniform, which is the case for a gas inside a volume in the present sensor, this may be simplified to

$$R ( \vec{r} ) = \frac{ρ}{M} \int \vec{r} d V ,$$

Where the integral ∫\vec{r}dV is the centroid or geometrical center of the volume V. Thus it is relevant to refer to the mass center of an air volume, and the air will have a uniform density the mass center will be defined by the shape of the volume.

In the abovementioned article by Kuusela et al it is discussed how an acceleration affects the pressure in the volume Kuusela, equation 62, page 469):

$$p ( x ) = {p}_{0} - {ρ}_{gas} ax$$

where p(x) is the pressure at a distance x from the mass center in the direction of the acceleration, p₀ is the equilibrium pressure, ρ_{gas} is the mass density of the gas and a is the acceleration of the volume. From this is follows that if you align the mass centers of the gas on the inside and outside of the membrane the pressure change cause by an acceleration will be the same on both sides of the membrane. This leads to a net zero force on the membrane and thus no sensitivity to vibrations or accelerations. The shape of the volumes may be balanced according to accelerations in one or more directions.

The preferred embodiment of the present invention thus provides a gas detector that provides noise cancellation in three dimensions, without limiting the size of the measuring or reference volumes, by balancing the shape and position of the gas volumes. The present invention also provides a photo [symbol] measurement volume having a high efficiency.

The invention is described below with reference to the accompanying drawings, illustrating the invention by way of examples.

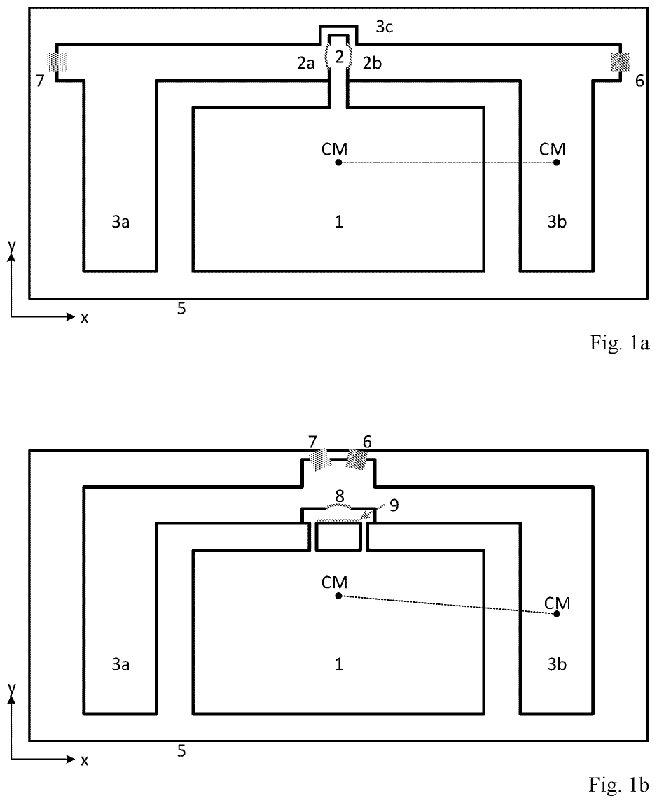

FIG. 1a,b illustrates schematically two embodiments of a gas detector according to the invention.

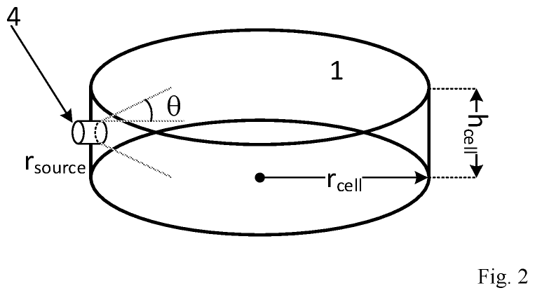

FIG. 2 illustrates the measuring cell according to a preferred embodiment of the invention.

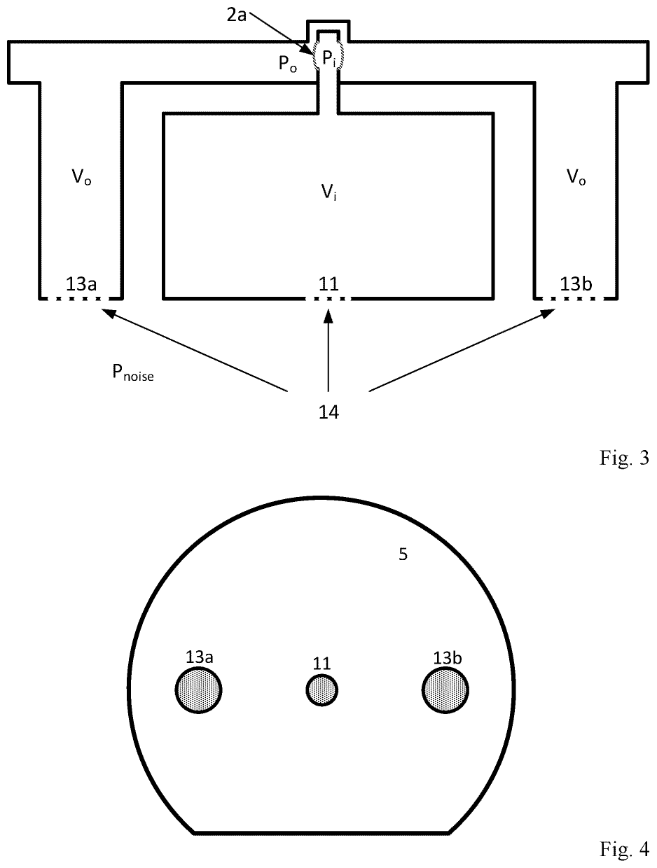

FIG. 3 illustrates a preferred embodiment of the gas detector according to the invention.

FIG. 4 illustrates the embodiment in FIG. 3 as seen from below.

As is illustrated in FIG. 1a the present invention relates to a photoacoustic gas detector 5 including a measuring cell or volume 1 with an interrogation unit 2 responding to pressure fluctuations in the measuring volume 1 by a gas flow passing freely between them. The interrogation volume is constituted by one or two membranes 2a,2b preferably with an optical readout unit 6, e.g. as discussed in WO2017/055219 and WO2017/089624 providing a measurement of the pressure fluctuations in the measuring volume 1 based on the distance or movements between the membranes. In the illustrated example the readout unit is an optical sensor 6 and a light source, preferably a laser or sufficiently coherent source 7, is positioned on the opposite side from the sensor 6. The readout unit being able to measure the distance fluctuations between the membranes 2a,2b, caused by the interference between the membranes. Other measuring means capable of measuring the pressure fluctuations may be contemplated, e.g. at least one of the membranes being an optical microphone, but in order to reduce sensitivity to vibrations in the x direction perpendicular to the surfaces a symmetric solution as discussed in WO2017/055219 and WO2017/089624 is preferred.

Reference volume 3a,3b is provided outside the interrogati [symbol] reference volumes 3a,3b are preferably combined by a channel 3c so as to provide one volume with even pressure on both sides of the interrogation volume. However, the channel 3c should preferably be sufficient to allow the gas in the reference volume 3a,3b move freely thus avoid building up pressure variations due to movements perpendicular to the membranes 2a,2b.

As illustrated the drawings show a cross section of the invention in the xy-plane with the z axis out of the plane of the drawing. Preferably the yz-plane between the surfaces 2a, 2b and the xy-plane normal to the surfaces 2a, 2b are planes of symmetry for the complete volumes 1, 2, 3a, 3b of the gas detector unit according to the invention. This way vibrations or movements in a direction x perpendicular to the surfaces will not affect the relative movements or distance between the surfaces 2a,2b, as discussed in WO2017/055219. Furthermore, there will not be a pressure gradient over the center of the surfaces 2a, 2b due to vibrations or movements in a direction z parallel to the surfaces.

In addition, the y-coordinate of the center of mass CM of the gas in each of the reference volumes 3a,3b and the measuring volume 1 including the interrogation volume2 should be equal. This way a movement or vibration in the y direction will not result in a pressure gradient of over the center of the surfaces 2a, 2b.

FIG. 1b illustrates an alternative embodiment corresponding to embodiment in FIG. 1a, except for the construction of the interrogation unit 2. In FIG. 1b the interrogation unit 2 is constituted by one moveable membrane 8 and a reflector 9 in the interrogation unit. The light source 7 and sensor 6 are positioned opposite from the measuring volume 1 and interrogation unit 2. As in FIG. 1a the back-volumes 3a,3b and measuring 1 are essentially symmetrical in the x and z directions, but since the membrane 8 is sensitive to movement in the y direction the y-coordinate of the center of mass CM of the measurement or back volumes have to be adjusted.

FIG. 2 illustrates the preferred embodiment of a measuring cell that may preferably be used in the present invention where the cell is shaped as a circular disc with a chosen radius r_{cell} and height h_{cell}.

As can be seen, a light source 4 is positioned in an opening with radius r_{source} in the wall defining the circumference around the disc shaped volume 1, the volume having a radius r_{cell} and height r_{cell}. The light source is chosen according to the absorption wavelength of the gas to be detected and is mounted in an opening in the wall. The inner walls of the cell are preferably covered with a reflective surface, e.g. Au, to increase the light intensity in the relevant wavelength range in the cell and thus the efficiency of the light absorption. The disk shape is also preferred in order to maximize the distribution of the light in the cell. The size r_{source} of the opening and light 4 source should be minimized so as to reduce loss.

FIG. 3 illustrates an embodiment of the invention also including openings 11,13a,13b from the environment into the reference and measuring volumes 1,3a,3b. A gas from the environment may diffuse or move into these volumes. Periodic pressure fluctuation is induced in the measuring volume, which provides a difference in pressure between Pᵢ, between the surfaces 2a, 2b, and Pₒ, in the reference volume 3a, 3b. The said pressure fluctuation is induced by focusing a light source with an appropriate wavelength spectrum into the measurement volume, pulsing the source, or preferably, tuning the center wavelength of the source to move in and out of the absorption spectrum of the gas inside the volume.

Preferably the openings 11,13a,13b are provided with sintered filters 14 to reduce acoustic noise Pₙₒᵢₛₑ while equalizing the pressure outside Pₒ and inside Pᵢ the interrogation volume 2, by acting as an acoustic low-pass filter. Furthermore, when the reference volume includes two equal and symmetric parts 3a,3b the acoustic resistance Rᵢ,Rₒ of the sintered filters connected to the different volumes 1,2,3a,3b should correspond to the volun

[symbol]

$$\frac{{R}_{i}}{{R}_{o}} = \frac{{V}_{o}}{{V}_{i}}$$

so that the pressure change outside and inside the interrogation volume 2 due to the acoustic noise is the same, effectively cancelling out the acoustic noise.

In FIG. 4 the gas detector is shown from below illustrating the relative size of the filters 11,13a,13b.

To summarize the present invention relates to a photoacoustic gas detector including a gas measuring volume and a gas reference volume. The volumes are separated by at least one flexible membrane being responsive to pressure differences between the volumes and the detector also comprises a light source emitting light in a predetermined wavelength range into the measuring volume. The wavelength range being is chosen based on the absorption spectrum of a gas to be measured so that the absorption leads to a temperature and thus pressure increase in the measuring volume which results in a movement in the membrane. The gas detector also includes a measuring unit for measuring membrane movements,

The measuring and reference volumes both define known mass centers, the position of the mass centers being chosen so as to balance the pressure on the membrane when subject to a movement in at least one predetermined first direction so that a movement in the detector in a direction will result in the same pressure variations on both sides of the membrane(s).

The gas measuring volume 1 is preferably constituted by a disk shaped volume with a predetermined radius r_{cell} and height h_{cell} enclosed in a container with a reflecting inner surface, preferably Au coated, where the light source 4 being mounted on the circumference of the disk emitting light into said volume. This way the light is distributed over the complete volume increasing the coverage and sensitivity of the detector.

The position of the mass centers may therefore chosen so as to balance the pressure on the membrane when subject to a movement in a predetermined first direction and second direction being perpendicular to the first direction.

The reference volume may be constituted by two symmetric volumes relative to the membrane(s) communicating through a channel between the volumes on each side of the membrane(s).

According to one preferred embodiment the detector includes two membranes, and the measuring volume includes an interrogation volume separating the membranes. The two membranes being symmetrically positioned along an axis being perpendicular to the first direction perpendicular to the plane defined by the membrane surfaces.

The measuring cell and the reference volumes have openings toward the environment for pressure equalization and for allowing gas enter into the volumes. The reference volume may contain the same gas as the measuring volume so that the only difference is the that the gas in the measuring volume absorbs the light emitted by the light source.

The reference volume V₀ may then be constituted by two connected volumes, each having an opening area Ab and the measuring volume having an opening area Am and the total volume of the measuring volume being Vᵢ, the sizes of said openings in the reference volumes, preferably with sintered filters, and measuring volume are chosen so that Rᵢ/R₀=V₀/Vᵢ where Rᵢ and R₀ are the acoustic resistance of the openings in the respective volumes Vᵢ and V₀, taking into account a possible sintered filter.

1-9. (canceled)

10. A photoacoustic gas detector including a gas measuring volume and a gas reference volume, the volumes being separated by at least one flexible membrane being responsive to pressure differences between the volumes, the detector also comprising a light source emitting light in a predetermined wavelength range into the measuring volume at a chosen rate for generating pressure fluctuations in the volume, the wavelength range being chosen based on the absorption spectrum of a gas to be measured, the gas detector also including an interference measuring unit for measuring distance fluctuations between the membranes caused by the pressure fluctuations, wherein the measuring and reference volumes both define known mass centers, the position of the mass centers being chosen so as to balance the pressure on the membrane when subject to a movement in at least one predetermined first direction y.

11. The gas detector according to claim 10, wherein the gas measuring volume is constituted by a disk-shaped volume enclosed in a contained with a reflecting inner surface, said light source being mounted on the circumference of the disk emitting light into said volume.

12. The gas detector according to claim 10, wherein the position of the mass centers being chosen so as to balance the pressure on the membrane when subject to a movement in a predetermined first direction and second direction being perpendicular to the first direction.

13. The gas detector according to claim 11, wherein the reflecting surface is Au coated.

14. The gas detector according to claim 10, wherein the reference volume is constituted by two symmetric volumes relative to the membrane(s) communicating through a channel.

15. The gas detector according to claim 10, comprising two membranes wherein the measuring volume includes an interrogation volume separating the membranes, the two membranes being symmetrically positioned along an axis being perpendicular to the first direction.

16. The gas detector according to claim 10, wherein the measuring and reference volumes have openings toward the environment for pressure equalization.

17. The gas detector according to one of claim 16, wherein the openings are provided with a sintered filter.

18. The gas detector according to claim 16, wherein the reference volume V₀ is constituted by two connected volumes, each having an opening area Ab and the measuring volume having an opening area Aₘ and the total volume of the measuring volume being Vi, the sizes of said openings in the reference and measuring volumes Vᵢ,V₀ being chosen so as to provide corresponding acoustic resistances Rᵢ,R₀ are chosen so that Ri/R₀=V₀/Vᵢ.

19. The gas detector according to claim 10, wherein the measuring volume is defined between two symmetric, moveable membranes, the measuring unit being configured to measure the distance fluctuations between the membrane, wherein a direction x perpendicular to the membrane surfaces is perpendicular to the predetermined first direction y.

20. The gas detector according to claim 10, wherein the light source is configured to emit light in a predetermined wavelength range into the measuring volume by tuning the center wavelength of the source to move in and out of the absorption spectrum of the gas to be measured at a predetermined rate.
